(19) United States
(12) Patent Application Publication
(10) Pub. No.: US 2010/0268658 A1
(43) Pub. Date: Oct. 21, 2010

(54) METHOD FOR COLLECTING, TESTING AND DISTRIBUTING MILK

(75) Inventors: Elena Medo, Murietta, CA (US); Martin Lee, Studio City, CA (US); David Rechtman, Hermosa Beach, CA (US)

Correspondence Address: COOLEY LLP;ATTN: Patent Group, Suite 1100, 777 - 6th Street, NW, WASHINGTON, DC, 20001, US

(73) Assignee: Prolacta Bioscience, Monrovia, CA (US)

(21) Appl. No.: 12/728,811

(22) Filed: Mar. 22, 2010

Related U.S. Application Data
(63) Continuation-in-part of application No. 11/679,546, filed on Feb. 27, 2007, now abandoned.
Application No. 11/679,546 is a continuation-in-part of application No. 11/526,127, filed on Sep. 22, 2006, now abandoned.
Application No. 11/526,127 is a continuation-in-part of application No. 12/372,376, filed on Feb. 17, 2009.
Application No. 12/372,376 is a division of application No. 11/012,611, filed on Dec. 14, 2004, now abandoned.
Application No. 11/012,611 is a continuation of application No. 10/144,325, filed on May 13, 2002, now abandoned.
(60) Provisional application No. 60/720,192, filed on Sep. 23, 2005.
(60) Provisional application No. 60/290,823, filed on May 14, 2001.

(51) Int. Cl. G06Q 10/00; G06F 17/00; C12Q 1/70; C12Q 1/04; G06Q 50/00

(52) U.S. Cl. 705/336; 235/375; 435/5; 435/34

(57) ABSTRACT

The disclosure is related generally to a method for facilitating the collection and distribution of milk over a computer nextwork among a milk provider, a milk company and a milk distribution center

### CROSS REFERENCE TO RELATED APPLICATIONS

This application is a continuation-in-part application of U.S. patent application Ser. No. 11/679,546, filed Feb. 27, 2007 which is a continuation-in-part application of U.S. patent application Ser. No. 11/526,127, filed Sep. 22, 2006, which claims priority to provisional application 60/720,192, filed Sep. 23, 2005. This application is also a continuation-in-part of U.S. patent application Ser. No. 12/372,376, filed Feb. 17, 2009, which is a division of U.S. patent application Ser. No. 11/012,611, filed on Dec. 14, 2004, now abandoned, which is a continuation of U.S. patent application Ser. No. 10/144,325, filed on May 13, 2002, now abandoned, which claims priority to provisional application 60/290,823, filed on May 14, 2001. The disclosure of each is incorporated herein by reference in their entireties.

### FIELD OF THE INVENTION

The invention is related generally to a method for monitoring, collection and distributing human milk.

### BACKGROUND

The lack of a standardized source of human milk, available for research purposes, has seriously hampered scientific investigation of human milk as well as the majority of the nearly 4,000 unique, species-specific milks. Accordingly, it would be desirable to provide a standardized source of human milk that can be modified to reflect the various stages of lactation and various immune responses.

Although the presence of immunoglobulins in human milk has been acknowledged for a number of years, the specific role and function of each of the human milk immunoglobulins and their subclasses has been poorly understood. For the patient of any age suffering from an immune disorder, the worldwide gamma globulin shortage is impacting care to such a degree that some patients are unable to obtain treatment. Purified immunoglobulins from human milk hold the potential for a solution for this worldwide shortage. Accordingly, it would be desirable to provide formulations comprising higher levels of immunoglobulins isolated from human milk, as well as methods to deliver these formulations to patients.

It has been known for a long time by physicians, scientists and nutritionists that the best food or nutrition supplied to an infant is its own mother's milk, i.e., fresh human milk. Recent research has indicated that “species-specific” milk plays a significant role in disease prevention and the severity of disease when the infant does become ill. Until recently, the reasons behind the superiority of species-specific milk were not well understood, nor were the various components and the roles they play in development and disease prevention. It is recognized, however, that many situations arise wherein the infant cannot obtain its mother's milk and as a result a suitable replacement is desired. Artificial baby milks, predominantly based on cow's milk, have been prepared and used to nourish an infant but there is increasing evidence that infants fed artificial baby milks suffer long-term ill consequences. It has been suggested that the exposure of an infant to any foreign proteins, such as the bovine protein, during the first few days of life will increase the infant's chance of becoming afflicted with juvenile diabetes. Other ill effects include allergies, lowered immunity, gastrointestinal disorders, respiratory disease and other associated etiology. Although much effort has been made to improve synthetic infant milk formulas, attempting to make them more closely simulate mother's milk, the presence of living organisms and other “species-specific” cells that act in a way to trigger other disease preventing mechanisms in the infant, these efforts have proven futile.

According to Jenness and Sloan, (1970) “The Composition of Milks of Various Species: a Review,” Dairy Science Abstracts 32(10):599-612, human milk contains three major groups of constituents that carry strong “species-specific” and “organ-specific” missions: (1) constituents specific to both organ and species, including proteins and lipids; (2) constituents specific to organ but not species, including lactose; and (3) constituents specific to species but not to organ, including albumin and some immunoglobulins.

Human milk is not a uniform body fluid; instead, it is a secretion from the mammary gland of constantly changing composition. In nature, the composition of human milk changes not only from day to day, but also throughout the course of a single day. While the reasons and outcome of these changes are not fully understood, it is intuitive to believe that these changes benefit the species and that substantial advantages may be gained for the infant who is provided an opportunity to reap the benefits of a modified formulation of 100% human milk. Accordingly, it would be desirable to provide formulations comprising human milk proteins as nutritional supplements and therapeutics for patients in need of gamma globulin therapy.

Unlike blood providers who give their donation under the direct supervision of the blood bank personnel, human breast milk providers tend to pump their milk at home or other locations convenient to them and then store the breast milk in their freezers until they have accumulated enough to bring to the milk collection center.

Generally, the milk providers need to be physically close to a hospital or other recognized milk collection/distribution center. This physical proximity of the providers to the collection center allows the collection center to screen the providers. It also provides a location where the providers can drop off the expressed milk in a timely manner.

Generally, the collection center also provides the milk to infants who require the milk. Thus the quantity of milk available to the infants is dependent on the number of providers located in the immediate vicinity of the collection center.

It would be advantageous if there was a method for collecting milk from providers physically located away from collection centers/hospitals as this would increase the population of providers. It would be advantageous if there was a central repository for the testing and distribution of the milk. This would allow the quality of the milk to be monitored and it would allow centers which require the milk to obtain the milk from a wider geographic region.

Thus, a method and system for efficient collection, demand, supply, delivery and payment for milk is needed to more evenly distribute the benefits and burdens of the industry.

### SUMMARY

This invention is directed to a method for facilitating the collection, delivery and supply for milk (e.g., human milk).

Compositions containing 100% human milk proteins, including the so-called host resistance factors (HRF) of human milk, as well as other nutrients, living cells, and components are useful when employed to enhance and improve outcomes for babies and children who are not able to obtain human milk from their mothers (or cannot obtain enough mother's milk or mother's milk in the formulation needed due to immunodeficiency of the mother) as well as other patients (including adults) suffering from immune disorders, nutritional disorders and other diseases and dietary challenges.

The sequential administration of many of the human milk constituents provides substantial value to the recipient because of metabolic and catabolic processes. It is at the core of the present invention to utilize such human milk constituents, in their processed form, in such a sequential fashion as to provoke the same type of chain reaction in the body. With this concept, the pairing of the processed milk tissue with the sequential and differentiated delivery methods, patients may enjoy a new type of preventative and therapeutic medicine. Because human milk immunoglobulins are specifically targeted to many diseases of the newborn, as well as the protective functions of the mucus membranes of the newborn's body, and contain higher levels of IgA, IgD, IgM and IgE, the term “panoglobulin” or “lactapanoglobulin” has been coined for this newly identified formulation. In addition to fighting immune disorders with a human-milk origin panaglobulin, patients preparing for surgery, chemotherapy, radiation or other “currently accepted, but destructive” therapies, may enjoy preliminary therapies that may mitigate the ill effects of their upcoming procedure. In the same fashion, the constantly changing nature of species-specific milk allows for the inclusion of the mammary gland as a laboratory of sorts, seeking not to simply initiate and artificially replicate structures like antibodies and proteins, but instead, to produce a bonafide human-produced fluid that can be isolated, processed and delivered for a highly targeted use against disease.

One embodiment of the present invention provides a nutritional formulation of isolated human milk containing protective human milk proteins or host resistance factors of human milk suitable for infant consumption which can be directly administered to an infant. In a further embodiment the nutritional formulation of isolated human milk further comprises human milk cells. In a still further invention, the human milk cells comprise lymphocytes and macrophages. In another embodiment, the human milk proteins comprise alpha-lactalbumin, beta-lactoalbumin, lactoferrin, serum albumin, lysozyme and secretory IgA.

Another embodiment of the present invention provides a method of isolating human milk comprising the steps of collecting a sample of human milk from a donor in a collection device, storing the sample of milk obtained from the donor, and processing the milk sample by conducting a nutritional analysis on the milk sample; fortifying the sample with heat-resistant nutrients, pasteurizing the sample; fortifying the pasteurized sample with heat-sensitive nutrients and testing the sample to ensure successful pasteurization. In another embodiment, the method of isolating human milk comprises the steps of collecting a sample of human milk from a donor in a collection device, wherein the collection device comprises a computer chip capable of recording temperature variations and handling conditions of the collection device; storing the sample of human milk obtained from the donor; and processing the sample of human milk from the donor, wherein the processing comprises the steps of: conducting a nutritional analysis on the sample of human milk; conducting a first fortification of the sample of human milk with heat-resistant nutrients; pasteurizing the sample of first fortified human milk; conducting a second fortification of the sample of human milk with heat-sensitive nutrients; and testing the sample of fortified human milk to determine whether the pasteurization was successful. In a further embodiment, the computer chip that is the collection device is further capable of recording the origin of the sample, contents of the collection device, volume of the collection device, shipping dates of the sample or lot number of the sample. In a further embodiment, the sample of human milk is frozen prior to the processing step. In yet a further embodiment, the collection device further comprises a twist-turn valve to open and release an amount of milk through a one-way valve into a test vial.

Yet another embodiment of the present invention provides a system for delivering human milk to an infant. The system contains a feeding tube treated to minimize adherence of milk fat to the interior of the feeding tube; a heated sheath surrounding the feeding tube and an enteral pump removably mounted to a motorized platform, the enteral pump capable of transferring human milk through the feeding tube from a storage container to the infant. In a further embodiment, the inside of the feeding tube is coated with silicone.

In one embodiment, the invention is directed to a method for facilitating the collection and distribution of human milk over a computer network among a milk provider, a milk company, and a milk distribution center comprising the steps of: qualifying, by said milk company, at least one provider to participate; providing a provider account code wherein said provider code corresponds to the qualification information for that provider, receiving filled first milk containers bearing the provider code from the provider; establishing a database for facilitating on-line display of a plurality of descriptive line items corresponding to, respectively, at least one of the filled milk containers, wherein each of said descriptive line items comprises an identification of the milk, including the quantity, quality and provider code; processing said human milk and filling second milk containers for shipment; and effecting the shipment of second filled milk containers to a distribution center.

It is contemplated that the method may further comprise the step of providing empty first milk containers to the provider.

It is contemplated that the method may further comprise establishing a relationship with a milk collection center. It is contemplated that the milk collection center may qualify the provider of the milk. It is further contemplated that the milk collection center may provide the qualification data directly to the milk company or to the database.

It is contemplated that the provider may ship the filled milk containers directly to the milk company. Alternatively, the provider may deliver or ship the filled milk containers to the milk collection center and the milk collection center will in turn ship the filled milk containers to the milk company. In one embodiment the unique code is a bar code. In another aspect, the milk is tested for viral and bacterial contaminants.

These and other aspects of the invention will become more evident upon reference to the following detailed description and attached drawings. It is to be understood however that various changes, alterations and substitutions may be made to the specific embodiments disclosed herein without departing from their essential spirit and scope. In addition, it is further understood that the drawings are intended to be illustrative and symbolic representations of an exemplary embodiment of the invention and that other non-illustrated embodiments are within the scope of the invention.

### BRIEF DESCRIPTION OF THE DRAWINGS

The subject of the invention will hereinafter be described in the context of the appended drawing figures wherein the numerals denote like elements or steps.

FIG. 1 illustrates the key to the symbols in the other figures in accordance with an exemplary embodiment of the invention.

FIG. 2 illustrates the system for initial interview screening of the female provider in accordance with an exemplary embodiment of the invention.

FIG. 3 illustrates the system for completed screening of female provider, provision of collection bottles and collection of expressed human breast milk in accordance with an exemplary embodiment of the invention.

FIG. 4 illustrates the system for transfer of milk to the company and review of the milk in accordance with an exemplary embodiment of the invention.

FIG. 5 illustrates the system for quality testing of the expressed breast milk, assignment of lot number and distribution to milk distribution center in accordance with an exemplary embodiment of the invention.

FIG. 6 illustrates the system for re-qualification of the human provider in accordance with an exemplary embodiment of the invention.

### DETAILED DESCRIPTION

Unless defined otherwise, technical and scientific terms used herein have the same meaning as commonly understood by one of skill in the art to which this invention belongs. All references cited herein are incorporated by reference in their entirety.

As used herein, “human-milk” means any stage of human milk production including the production of breast secretions not associated with lactation. These stages include, but are not limited to, colostrums, transitional milk and mature milk.

As used herein, “species-specific milk” means any milk that would be processed or formulated to provide an advantage of any kind to its own offspring.

As used herein, “second-best species-specific milk” means any milk that would be processed or formulated to provide a “close second” to its own species-specific milk resulting in better outcomes than using the standard bovine or soy based milk replacer.

The “milk collection center” is a location where the milk providers or mothers can deliver the milk. The milk collection center is also the location where the providers or mothers can be initially screened for qualifications for providing the milk. The milk collection center may be a hospital, or a local health clinic or other health facility. It is contemplated that the milk collection center may be one center for the initial screening and a different center for the delivery of the milk. It is not required that the milk collection center remain the same location or physical or company entity throughout the process.

The “mother” or “provider” is the lactating mother who has offered to provide her expressed milk for this purpose. It is contemplated that the milk will be donated, but the milk may be purchased. The “mother” becomes “qualified” to provide the milk following biological testing and identifying characteristics of the donor during the process of this invention.

A “milk company” is the company or hospital that coordinates the process of the invention. The milk company maintains the database or has the database maintained. The milk company may receive and ship the milk. It is contemplated that the milk company may arrange for the milk to be provided, collected and shipped or monitored by the methods of this invention.

A “reference laboratory” is a laboratory for conducting testing of biological samples and/or milk samples. The reference laboratory for conducting tests on the biological samples may differ from the laboratory used to conduct tests of the milk samples. Furthermore the “reference laboratory” may be the milk company or hospital.

A “milk distributor” is a location which will accept the provided milk and distribute it to persons requiring the milk. Exemplary embodiments of a milk distributor are a hospital or milk bank or health clinic or other recognized distributor of human milk.

One skilled in the art will recognize many methods and materials similar or equivalent to those described herein, which could be used in the practice of this invention. Indeed the invention is in no way limited to the methods and materials described herein.

The present invention describes a method that includes multiple steps and processes to harvest or isolate, store, transfer, process, package and deliver a variety of pharmaceutical and nutritional formulations containing cells and tissues comprising 100% human milk tissue, fractionated human milk tissue components and specifically reunited compounds, as well as novel methods and procedures to affect levels of such fractionated human milk tissue components, isolate them from raw human milk and deliver them through various methods including (but not limited to) ingestion, inhalation, intra-nasal administration, eye drops, ear drops, enema, douche, lavage, transdermally, rectally, intravenously, intramuscularly injection, direct injection, direct topical application, ng tube and jg tube.

Additionally, these formulations may be delivered through any of these methods, but when delivered, the present invention describes a sequence of delivery by which certain components or compounds will catabolize to create optimum conditions for the sequential delivery of an additional compound. For instance, if the formulation is nutritionally focused, the present invention provides a formulation that is specifically delivered in the morning, with a different formulation delivered in the afternoon and evening.

Little is understood at this time, as to why the formulation of mammalian milk evolves throughout the day. The present invention is directed to a method by which this differentiation is preferred and would create an improved outcome for the patient. Additionally, by any method, there may be an advantage to the “priming” of the patient by the delivery of certain processed human milk components, thereby eliciting a response in the patient's body that will improve outcomes when the next treatment in the sequence is followed. This sequential treatment concept would not be limited to the method of delivery. Instead, the present invention relates to the possibility that multiple delivery methods may actually trigger multiple advantageous responses in the patient, increasing the patients' chance of an improved outcome by coaxing the patient's system into active collaboration with the treatment method. This method simulates the natural processes of the mammalian immune system, which cannot be described as any one “silver bullet” but a series of complex communications between multiple cell structures and the offending pathogen.

The present invention relates to use of the disclosed methods and formulations from all mammalian species and is not limited to human beings. Additionally, the present invention encompasses all breast fluids as a potential source for harvesting milk and immune cells, as the mammary gland is a lymphoid organ, capable of producing immunoglobulins with or without accompanying lactation.

In general the invention includes a unique method for screening milk providers, collecting milk (e.g., expressed human breast milk), testing the milk and distributing the milk to distribution centers. The system communicates with and takes advantage of existing collection, transportation, tracking, distribution and banking systems to increase the collection, testing and distribution of the human breast milk. The interactive database includes real-time information in connection with a transaction.

The invention enhances extranet functionality by substantially reducing the providers, collection agencies and distributors actions in the milk process by providing an integration of systems to maximize convenience and efficiency. In general, the invention performs various functions for the providers, collection centers and distribution centers thereby allowing real-time transactions. For example, the invention pre-qualifies providers, provides databases and financial relationships to assist in the collection of milk.

In another aspect, the invention provides data regarding a mother's milk composition including, but not limited to, fat content, protein content and the like. For example, in a hospital setting a mother may wish to use her own milk to feed a newborn in the hospital. In such instances, the milk can be expressed by the mother, collected, tracked, analyzed and modified to adjust the caloric, protein, or other content of the milk and then distributed to the mother's own child.

The system provides connections to transportation systems to enhance scheduling and tracking of products. In an exemplary embodiment the present system communicates with a shipper's (e.g., Federal Express and/or United Parcel Service) system or courier service and database to share its credit information, shipping information, tracking information and the like thereby enhancing the functionality of the system. The system provides a database for tracking the qualifications of a subject provider (e.g., a human provider) and the quality of the milk.

In one embodiment, the invention pertains to a system of milk collection and distribution running over the a network of computers (e.g., the Internet). It will be appreciated however, that many applications of the invention could be formulated. One skilled in the art will appreciate that the network may include any system for exchanging data or transacting business, such as the Internet, an intranet, an extranet, WAN, LAN, satellite communications, and/or the like. The users may interact with the system via any input device such as a keyboard, mouse, kiosk, personal digital assistant, handheld computer (e.g., Palm Pilot®), cellular phone and/or the like. Similarly, the invention could be used in conjunction with any type of personal computer, network computer, workstation, minicomputer, mainframe, or the like running any operating system such as any version of Windows, Windows NT, Windows XP, Windows 2000, Windows 98, Windows 95, MacOS, OS/2, BeOS, Linux, UNIX, or the like. Moreover, it will be readily understood that the invention could be implemented using TCP/IP communications protocols, IPX, Appletalk, IP-6, NetBIOS, OSI or any number of existing or future protocols.

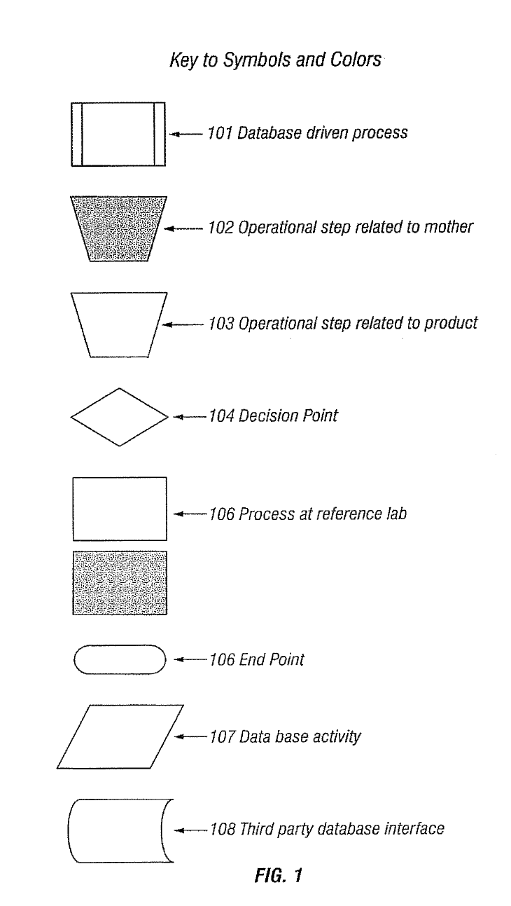

More particularly, with respect to FIG. 1, various types of operations are shown. The various operations include 101 database driven process; 102 operations related to the mother or provider; 103 operations related to the donated milk product; 104 decision point; 105 process step at the reference laboratory; 106 end point; 107 database activity; and 108 third party database interface. The third party could be any party given access to the database. Such access may be by way of the internet or other means known in the art. Such access may be limited to entry of data into the database or access to limited information from the database. Possible third parties include the milk company, the milk collection center, the milk distribution center, the reference laboratory, and the milk provider. For example, the milk collection center may be able to enter the information regarding the provider and the number of milk containers provided to the mother and collected from the mother and the quality of the milk. The reference laboratory may be able to enter the results from the biological tests conducted.

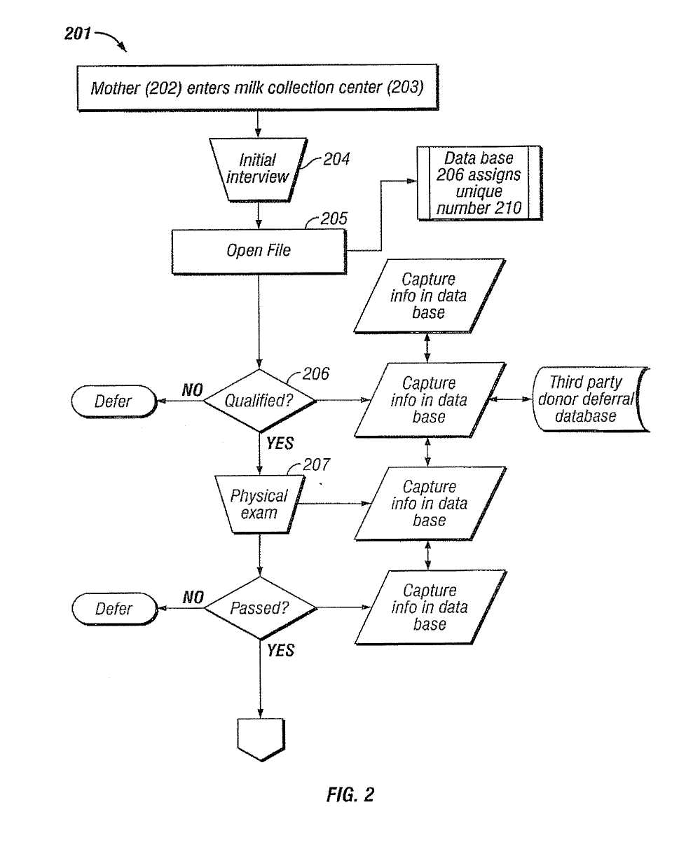

Referring now to FIG. 2, a system 201 for initial interview screening of a female provider is provided in accordance with an exemplary embodiment of the invention. System 201 includes a mother or milk provider 202 and a milk collection center 203. Interactive database 206 provides an on-line database for the various parties to access to assist in carrying out a transaction.

For example, interactive database 206 may be stored on a server such that each party may access interactive database 206 via a web site for on-line interactive communication. of course, interactive database 206 may be implemented in any number of communication systems, including wireless communication, packet switched networks (e.g. via an Internet service Provider (ISP)), wired communication, intranet, extranet, and the like. To further illustrate the variety of communication media that interactive database 206 may be implemented in laptops, cellular phones, personal digital assistant (PDA) technology, modems, and the like may be used.

To understand an exemplary method of conducting business using system 201, FIG. 2 also illustrates the various steps in a transaction. The putative provider 202, contacts the milk collection center 203. The milk collection center 203 interviews and/or test the potential milk provider 202 to determine whether the provider 202 would be suitable as a milk provider (204). The milk collection center 203 opens a file (205) for the milk provider 202 and the database 206 assigns a unique provider code 210 to the milk provider 202. In some aspect, the same unique provider code will be assigned to a child of the donor to facilitate matching the donated mother's milk to the child. If the milk provider 202 passes the initial qualification screening (204) this result is captured into the database 206 under the unique provider code 210.

Technicians of the milk collection center 203 or doctors conduct a physical examination (207) of the milk provider 202. This physical examination may be conducted at this time or may have been conducted at an earlier date and the health of the provider certified by a physician. If the milk provider 202 passes the physical examination, this result is captured into the database 206 under the unique provider code. If the milk provider 202 does not pass either the initial interview or the physical examination, the provider is rejected.

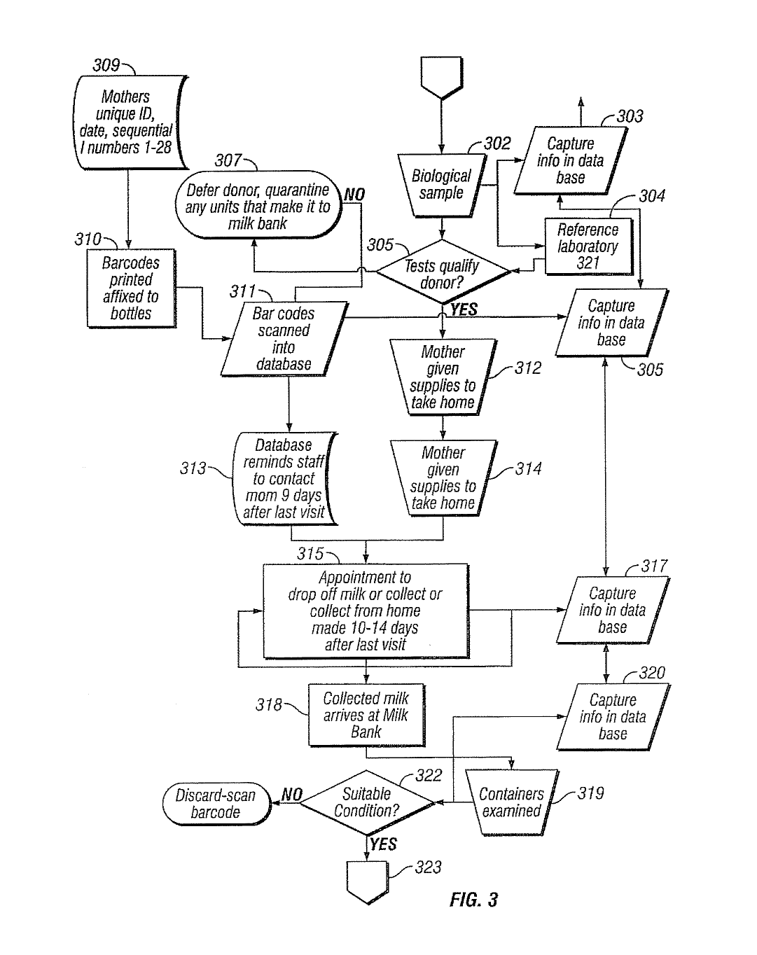

Turning now to FIG. 3, if the milk provider 202 passes both the initial interview and the physical examination, biological samples are taken from the provider 202 (302). An indication that biological samples were drawn is entered into the database (303). The biological samples are sent to a reference laboratory 321 for analysis (304). The laboratory 321 analyzes the biological samples and the results are entered into the database 206 under the provider's unique code 210 (305). The reference laboratory 321 may have direct access to the database 206 for entering the information or they may report the information to the milk company for entry into the database. Such reporting may be in paper or electronic form. In one aspect, the biological sample is the expressed milk.

The biological tests which are conducted may include viral and biological analysis known in the art. Such tests may include testing for human viruses such as HIV1/2, HTLVI/II, HBV, HCV, and the like. Such tests may include testing for bacterial diseases such as syphilis and tuberculosis. In addition, the expressed milk can be tested for caloric content, protein content, IgG content, and may include a panel of test for drugs and pathogens.

Such testing may also include identification of unique identity markers to identify the provider, such testing may include nucleic acid testing or peptide or antibody testing. The disclosure of U.S. Ser. Nos. 60/719,317, filed Sep. 20, 2005, and 60/731,428, filed Oct. 28, 2005, and International Application PCT/US2006/036827, entitled “Method of Testing Milk,” filed Sep. 20, 2006 are incorporated herein by reference.

If the biological tests disqualify the milk provider 202 as a provider, all milk provided by that mother is quarantined and destroyed (307). If the biological tests qualify the milk provider 202 as a provider, labels 309 are printed with the provider's unique code and affixed to the milk containers (310). The unique codes on the containers are entered into the database (311). In one aspect, the donated milk is analyzed for caloric content or protein content. Where the milk does not meet an optimal range of caloric content or protein content, the milk may be concentrated or modified by addition of a human milk fortifier to provide a target of about 3-10 kcal/oz (e.g., about 4, 6, 8, or 10 kcal/oz) and about 1-6 grams of protein/100 ml (e.g., about 2.1, 2.5, 2.9 or 3.4 grams/100 ml). The expressed or modified milk product will then be returned to the mother or distributed to a neonatal unit for administration to the mother's infant. The correct infant is matched with the milk using a code on the bottle and a code associated with the infant or mother.

The milk containers may be bottles or bags. The bottles may be glass or plastic. The provider 202 takes the labeled bottles home (312). On an as needed basis, the provider (202) expresses her milk into the milk containers and freezes the containers containing the milk (314). It is contemplated that step 314 could occur a number of times in a day and/or on a daily basis.

The database 206 sends a timed reminder to the milk company to contact the provider 202 at an appropriate interval after the last visit to the collection center 203 to schedule pick-up or delivery of the filled milk containers (313). This interval can be at least 5 days, at least 7 days, at least 9 days, at least 11 days, and/or at least 14 days. The days may be calendar days or business days after the provider's 202 last visit. It is contemplated that the milk will be collected in sufficient time to maintain the quality of the milk.

The milk company or hospital contacts the provider and arranges an appointment to collect milk (e.g., previously expressed and frozen stored milk containers) (315). The filled milk containers may be delivered by the provider 202 to the milk collection center or hospital 203, or the milk may be collected using a freight service (318). The number of containers of milk delivered to the milk collection center 203 is recorded in the database 206 (317). The filled milk containers are examined (319)and the condition of the containers recorded in the database 206 (320). If in suitable condition the milk containers are shipped to the milk company (323). If the containers are not in suitable condition the containers and milk are discarded (322).

It is contemplated that the milk containers may be directly shipped to the milk company rather than to the milk collection center 203 by the freight company.

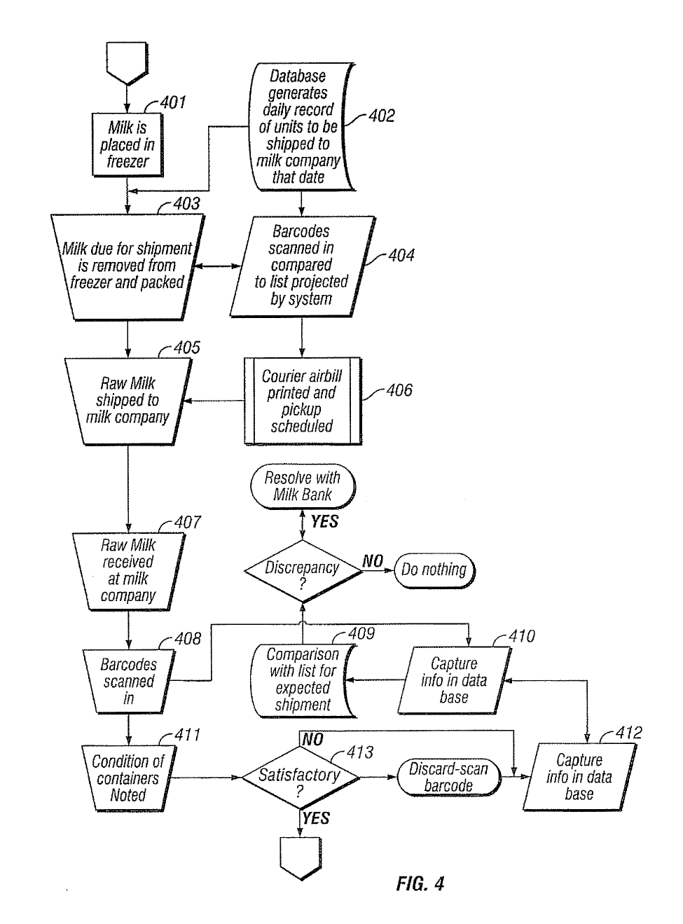

Turning now to FIG. 4, the milk can be placed in the freezer at the milk collection center (401). The database 206 generates a record of the units to be shipped to the milk company each day (402). The milk due for shipment is removed from storage (e.g., from a freezer) and the unique provider codes entered into the database (404). The milk containers to be shipped are packed for transport internally or externally (403). A courier airbill or bill of lading or transport notice is printed and shipment by a freight courier or other method is scheduled (406). The milk containers are shipped to the milk company by a courier (405).

The milk containers are received at the milk company (407). The unique provider codes are scanned into the database 206 (408) and compared to the list for shipment. (409 and 410) The condition of the milk containers is noted (411). If the condition of the milk container is not acceptable, the label 309 is removed, the condition is entered into the database and the milk discarded (413).

If the condition of the milk container is acceptable, a sample of the milk is obtained from each container (501). It also is contemplated that not every milk container from each provider will be sampled, rather it is contemplated that a representative number of milk containers may be sampled and tested. It is also contemplated more than one sample could be taken. The taking of the sample is recorded in the database 206 (502). Methods of obtaining a sample of the expressed milk include a stainless steel boring tool used to drill a core the entire length of the container Alternatively, a sample may be scraped from the surface of the frozen milk or pipetted by sterile pipette. The container may contain a separate portion which collects a sample of the expressed milk and this may be removed as the sample. The milk may be thawed and a sample isolated by pipette or other means.

It is contemplated that some samples may be pooled for the testing step. It is contemplated that pooled samples may be tested for presence of drugs or the presence of bacterial or viral contamination or caloric/protein content.

The milk sample is sent to the reference laboratory 321 for testing. This is recorded in the database 206 (502). The sample is tested at the reference laboratory for donor identity (505). It is contemplated that other testing for bacterial or viral infection and or presence of drags may also occur. In some aspect, the milk composition including, but not limited to, fat content, caloric content, protein content and like nutritional information may be analyzed.

The milk container is placed into quarantine pending the lab test results from the reference laboratory (504). If the milk sample is confirmed to originate from the provider 202 and there are no contaminants, the containers corresponding to the accepted sample are removed from the freezer for further processing (509 and 510). This information is stored in the database 206 and the provider 202 remains qualified (512).

If the milk sample does not pass the tests, the milk containers are discarded and the provider milk provider 202 indicated as disqualified (506). This information is captured in the database 206 (507).

The acceptable milk containers are sent for manufacturing and processing (511 and 513). It is contemplated that the milk after manufacturing and processing may be further tested for the presence of bacterial or viral contamination.

A lot number is assigned to the processed milk and attached to the final or second milk containers (514 and 515). The finished product (second filled milk containers) is placed in the freezer (516). The distribution center's (518) shipping data is printed on a label and affixed to the milk product (519). The information is entered into the database 206. (517) The product is shipped to the distribution center 518 by freight carrier (520).

In some aspect, where the nutritional content of the milk is tested, the nutritional content is entered in to the database and may be printed and associated with the collected bottles or lots. In some aspects, the nutritional content is modified and the modifications noted in the database and on the bottle or lot.

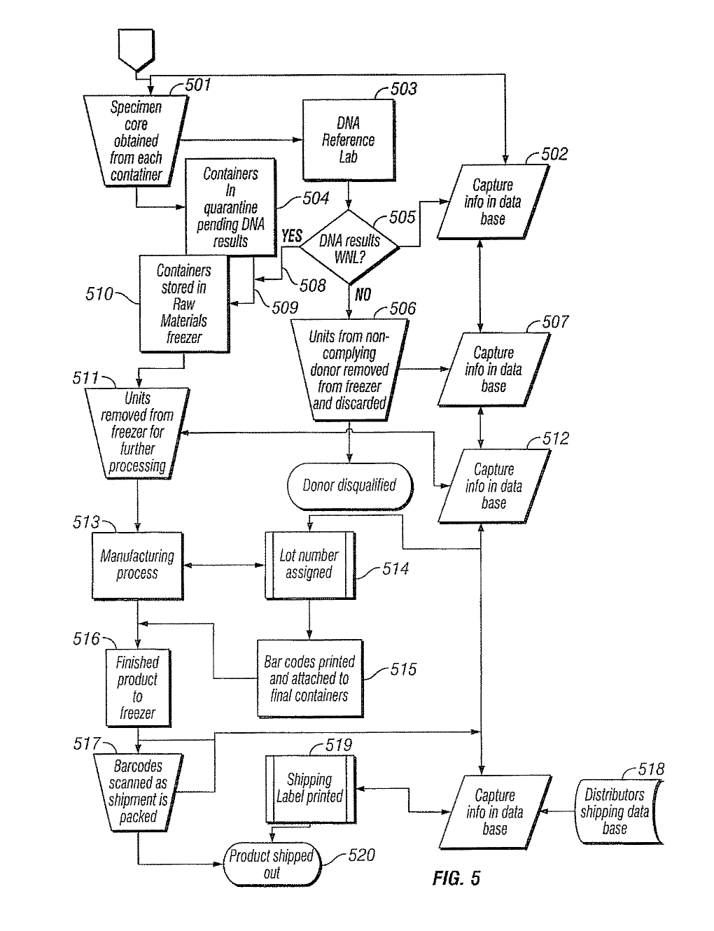

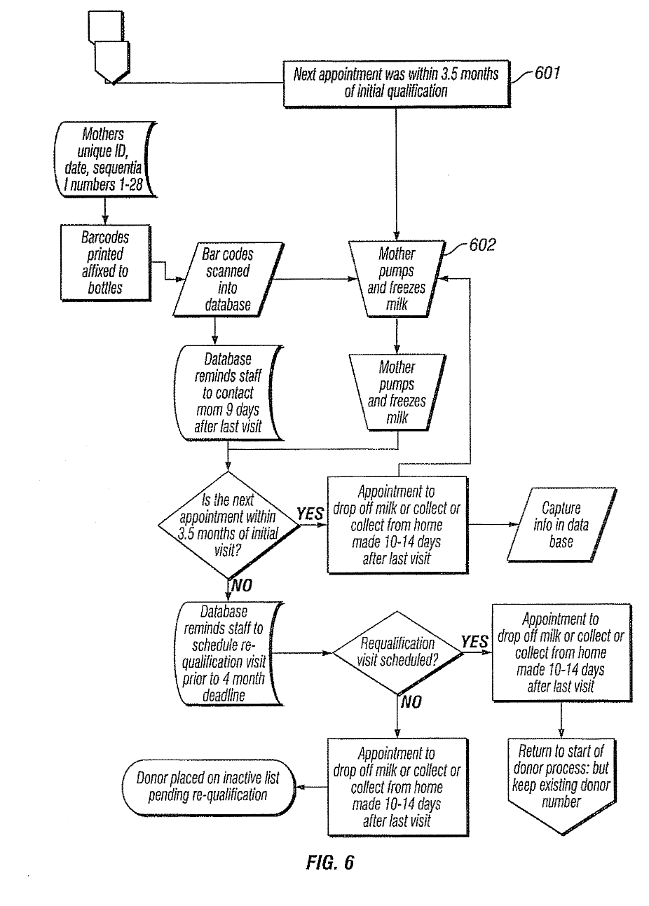

Turning now to FIG. 6, If the provider 202 passes the initial qualification, the database schedules another qualification appointment. This subsequent qualification appointment is scheduled for at least 1 month from the initial appointment, or at least 2 months from the appointment or at least 3 months from the appointment or at least 4 months from the appointment (601). The Provider 202 repeats all of the steps in FIGS. 2 and 3 up to 308. The provider 202 is given empty milk containers having the providers unique identity code (602) and the process set forth in FIGS. 3-5 is repeated until the provider fails qualification or decides to leave the program.

The various techniques, methods, and aspects of the invention described herein can be implemented in part or in whole using computer-based systems and methods. Additionally, computer-based systems and methods can be used to augment or enhance the functionality described herein, increase the speed at which the methods can be performed, and provide additional features and aspects as a part of or in addition to those of the invention described elsewhere in this document. Various computer-based systems, methods and implementations in accordance with the above-described technology are presented below.

A processor-based system can include a main memory, such as random access memory (RAM), and can also include a secondary memory. The secondary memory can include, for example, a hard disk drive and/or a removable storage drive, representing a floppy disk drive, a magnetic tape drive, an optical disk drive, flash drive, and the like. The removable storage drive reads from and/or writes to a removable storage medium. Removable storage medium refers to a floppy disk, magnetic tape, optical disk, and the like, which is read by and written to by a removable storage drive. As will be appreciated, the removable storage medium can comprise computer software and/or data.

In alternative embodiments, a secondary memory may include other similar means for allowing computer programs or other instructions to be loaded into a computer system. Such means can include, for example, a removable storage unit and an interface. Examples of such can include a program cartridge and cartridge interface (such as the found in video game devices), a movable memory chip (such as an EPROM or PROM) and associated socket, and other removable storage units and interfaces, which allow software and data to be transferred from the removable storage unit to the computer system.

The computer system can also include a communications interface. Communications interfaces allow software and data to be transferred between computer system and external devices. Examples of communications interfaces can include a modem, a network interface (such as, for example, an Ethernet card), a communications port, a PCMCIA slot and card, and the like. Software and data transferred via a communications interface are in the form of signals, which can be electronic, electromagnetic, optical or other signals capable of being received by a communications interface. These signals are provided to communications interface via a channel capable of carrying signals and can be implemented using a wireless medium, wire or cable, fiber optics or other communications medium. Some examples of a channel can include a phone line, a cellular phone link, an RF link, a network interface, and other communications channels.

In this document, the terms “computer program medium” and “computer usable medium” and “computer readable medium” are used to refer generally to media such as a removable storage device, a disk capable of installation in a disk drive, and signals on a channel. These computer program products are means for providing software or program instructions to a computer system.

Computer programs (also called computer control logic) are stored in main memory and/or secondary memory. Computer programs can also be received via a communications interface. Such computer programs, when executed, enable the computer system to perform the features of the invention as discussed herein. In particular, the computer programs, when executed, enable the processor to perform the features of the invention. Accordingly, such computer programs represent controllers of the computer system.

In an embodiment where the elements are implemented using software, the software may be stored in, or transmitted via, a computer program product and loaded into a computer system using a removable storage drive, hard drive or communications interface. The control logic (software), when executed by the processor, causes the processor to perform the functions of the invention as described herein.

In another embodiment, the elements are implemented primarily in hardware using, for example, hardware components such as PALs, application specific integrated circuits (ASICs) or other hardware components. Implementation of a hardware state machine so as to perform the functions described herein will be apparent to person skilled in the relevant art(s). In yet another embodiment, elements are implanted using a combination of both hardware and software.

In another embodiment, the computer-based methods can be accessed or implemented over the World Wide Web by providing access via a Web Page to the methods of the invention. Accordingly, the Web Page is identified by a Universal Resource Locator (URL). The URL denotes both the server machine and the particular file or page on that machine. In this embodiment, it is envisioned that a consumer or client computer system interacts with a browser to select a particular URL, which in turn causes the browser to send a request for that URL or page to the server identified in the URL. Typically the server responds to the request by retrieving the requested page and transmitting the data for that page back to the requesting client computer system (the client/server interaction is typically performed in accordance with the hypertext transport protocol (“HTTP”)). The selected page is then displayed to the user on the client's display screen. The client may then cause the server containing a computer program of the invention to launch an application to, for example, perform an analysis according to the invention.

A number of embodiments have been described. Nevertheless, it will be understood that various modifications may be made without departing from the spirit and scope of the description. Accordingly, other embodiments are within the scope of the following claims.

### EXAMPLES

The following examples are intended to illustrate various embodiments of the present invention and are not to be construed as limiting the scope of the invention.

### Example 1

### Gamma Globulin Formulations

At the center of this invention, is the intent to solve the worldwide shortage of gamma globulin. The current source of gamma globulin is blood serum, and specifically IgG from human blood. The present invention discloses a prophetic inclination, based upon a 15-year study of human milk, that a new form of gammaglobulin referred to herein as “panaglobulin,” “mammaglobulin” or “lactopanaglobulin” may replace the current gamma globulin. Because higher levels of IgA and IgM are present in human milk and colostrums, and a more diverse form of IgG as well, panaglobulins may provide protection beyond the scope of current gamma globulin therapy. Manipulation of the levels of immunoglobulins and their subclasses will result in formulations that are targeted at specific diseases or organ systems, making it possible to attack disease using nature's pharmaceutical laboratory, the mammary gland. Furthermore, milk donors who have weaned their babies or have initiated lactation without pregnancy could feasibly become human labs, becoming exposed through any method to mild strains of disease and producing the appropriate antibody in their milk. Since the breast is reactive to new exposures of pathogens, an array of new immunities can be produced to combat such diseases. Whether these types of donors could produce enough milk to become a primary source remains to be seen, but at least these donors could provide a human lab for biosynthesizing disease specific antibodies that could be replicated later using other methods.

Colostrum contains high levels of immunoglobulins, a vital defense mechanism that protects the newly born. sIgA provides immediate protection to the infant by lining the gastrointestinal system and providing a first defense against dangerous pathogens like E. coli and other devastating disease organisms. The invention discloses concentrated, processed sIgA for use as a prevention or therapeutic for gut disorders in patients of all ages. Potency levels will depend upon the severity of the disease, the general health of the patient and the cost of the processing.

Colostrum also contains IgG1, G2, G3, G4, IgM, IgD and trace amounts of other human origin immunoglobulins. All of these immunoglobulins function in a myriad of ways, targeting specific organs and disease states. Because the mammary gland is a lymphoid organ, it is capable of synthesizing immunoglobulins, especially the four IgG subclasses, making it possible to achieve a higher level of IgG subclasses in breast fluid than is present in human blood serum. This capability of the mammary gland is not limited to lactation, with measurable quantities of IgG present in breast ductal fluid from non-lactating women. Expressing ductal fluid may provide protective advantages to the donor, specifically the cleansing of the breast ductal system, as disclosed in a prior patent application by the inventor. This invention envisions breast fluid from non-lactating women as a potential source of human immunoglobulins. Current research cites a wide variety of volume and constituents present in colostrums, transitional milk and mature milk but little information exists. for the constituents present in the breast ductal fluid of non-lactating women. The present invention is directed to the ability to influence the volume and constituents of breast ductal fluid through dietary and pharmaceutical manipulation.

For nutritional and pharmaceutical applications, other valuable proteins contained in human milk include alpha-lactalbumin, beta-lactoglobulin, lactoferrin, serum albumin, lysozyme, and other proteins as well. Human milk has a higher proportion of alpha-lactalbumin and the host resistance factors or anti-microbial proteins of human milk, which include lactoferrin, lysozyme and secretory IgA, and account for 75% of the protein in human colostrum as compared with 39% in mature human milk. Additional human milk cells that provide substantial disease resistance in the newly born include lymphocytes, macrophages, and secretory IgA. Lactoferrin is present in relatively high amounts in human milk as is lysozyme and bifidus-stimulating factors. A major objective of this invention is to provide techniques and routines for improving the diet and feeding of infants, particularly very-low-birth-weight infants. By varying the levels of many of these species-specific milk constituents, the invention will result in a myriad of formulations specially suited to a wide variety of medical conditions.

### Example 2

### Collection of Donor Milk

U.S. Pat. No. 4,772,262, which is hereby incorporated by reference in its entirety, is directed to technology for milk removal. As disclosed in that patent, milk yields increase due to the sensory stimulus provided by the patented breast pump equipment. When milk yield increases, the formulation of milk including many of the valuable immunoglobulins also increase along with living cells, such as macrophages and lymphocytes. Lipids also increase and the mother's body responds to the stimulus by producing higher levels of prolactin that will trigger continuing milk supply and the secretion of additional nutrients into her milk.

### Example 3

### Storage of Donor Milk

Previous methods of collecting donor milk failed to recognize the importance of stimulation to the mammary gland as well as collection chambers designed for the anaerobic collection and transfer of donor milk. The invention describes such a method as part of its unique collection, storage and transfer system. Additionally, the preservation of milk components and nutrients is paramount to the success of the invention wherein harvesting of milk cells specific to the species will result in pharmaceutical and nutritional improvements in outcomes for the newly born or immune compromised patient. For that reason, it is important that the container in which the donor milk is stored, will preserve and protect these vital milk constituents from harm due to ultraviolet light and other damaging light rays. A UV coating or additive, applied to the collection bottle during the molding process or afterwards as an exterior coating or sheath will ensure that light degradation does not occur.

Finally, the design of the donor milk collection bottle should make it easy to draw off a sample of the donor milk without compromising the integrity of the milk sample. A proprietary design allows for a twist-turn valve to open and release a small amount of donor milk through a one-way valve into a test vial. The one-way valve prevents any bacteria or other pathogen from contaminating the milk sample. Additionally, a “tear down” design will allow frozen milk to be processed immediately, without the necessity of waiting for the milk to thaw. The tear down feature will provide an easy pull-tab that will strip the container from the frozen block of donor milk. The pull-tab will feature a tag on which a bar code is attached, so that during the tear down process, a “lot” numbering system will track the pooled milk back to their original donors.

### Example 4

### Transfer of Donor Milk

Novel designs for refrigerated transfer units utilizing alternative forms of energy and equipped with temperature indicator recorders ensure that the milk has been maintained under safe conditions. A programmable chip that records temperature variations as well as handling conditions (rough treatment can compromise milk quality by breaking cell walls), prevents the opening of the transfer case upon arrival at the processing plant. The milk is automatically rejected for quality issues and quarantined for further scrutiny. The transfer unit will contain a programmable chip that stores the contents, origin of contents, date shipped, date received, lot numbers and any other information required for quality control, regulatory or other reasons.

### Example 5

### Hospital Based Testing and Processing of “Mothers Own” Milk

The invention discloses a total quality control system that encompasses both routine and novel procedures and tests. For mothers wishing to provide their own milk for use specifically with their own baby, onsight testing will be done at the hospital. Standard donor screening will be done in accordance with current recommendations and accepted practices. In the present day, no routine testing is done in this case, and frequently the lack of testing causes consternation and concern in the physician with the end result being that babies are being routinely deprived of their mothers' milk. Upon questioning the areas of concern, several neonatologists indicated a concern for the presence of street drugs, disease pathogens and contaminants. To answer this concern, the invention includes a series of quick tests, designed to screen for the presence of the most common pathogens, drugs and contaminants. In order to provide the most efficient form of testing, a series of pumped milk is pooled, mixed and tested. A report is provided to the neonatologist and also placed in the infant's chart. The mothers' milk, intended for her own baby, is housed in the milk laboratory, under optimum storage conditions. Again, a temperature indicator on each container of milk ensures that milk has not been exposed to adverse conditions that may cause degradation or contamination. The temperature indicator is attached to a disposable cap that covers the container. In the event of adverse circumstances, the temperature indicator activates a locking mechanism and the milk is quarantined until further analysis can be done.

### Example 6

### Onsight (Hospital Based) Delivery Methods for Mothers Own Milk

Of special concern in high risk neonatal units, is the loss of milk fat through feeding tubes used to feed very-low-birth-weight, sick or pre-term infants. A special design for extruded tubing employs a method during the manufacturing process, that will eliminate the problem of fat sticking to the inside of the tubing. After extrusion, a heat treatment is applied to the inside of the tubing, via a “pull-through” rod. A heated element, coupled with an anti-static element, of sorts, eliminate the static charge while smoothing the “tackiness” of the interior tubing wall. Coupled with a heated sheath, used during the tube feeding to keep the flow of milk warm, the fat loss can be substantially decreased. A gentle rocking motion, created by a motorized platform on which the enteral pump sits, provides constant agitation and prevents the pumped milk from separating. Additional design features prevent the fat from clinging to the inside of the enteral syringe, in which the pumped milk is contained. A Teflon coating, or alternatively a silicone interior bag or collapsible bag made from a food safe polyvinyl may create additional solutions to this problem. Techniques associated with sequential feeding methods may also mitigate the problems associated with single feed method. By utilizing sequential feeds, the “foremilk” formulation (simulating the composition of the first milk a baby receives during a direct feeding from the breast), is administered. Low in fat, but high in volume, this feed usually takes more time than the higher fat “hindmilk” feed. The hindmilk feed can be then administered from a push syringe specially designed to conserve a large amount of the fat that normally would have stayed in the long tubing associated with the earlier feed.

Example 7

### Plant Processing Methods

In the practices of this invention the human milk proteins, including the so-called host resistance factors (HRF) of human milk, are prepared by chemically fractionating the same using standard techniques, such as the Cohn method, from pooled donor milk. This method will form the basis for the extraction of the immunoglobulins for the ultimate purpose of purification and processing into nutritional, IV and injectable forms. The present invention discloses a completely closed system for processing.

Under this system, there is no opportunity for contamination. When the donor milk is received at the processing center, a representative sample from each donor lot is tested and cultured. The remaining samples in the lot are transferred to the freezer to hold until the cultures are read. From the strip-down phase to the spray drying of the final product, all processing occurs within a sealed system. After the lot has been cleared for processing, the frozen containers of milk are placed in an anaerobic chamber where the strip down of the bottle occurs. A filter prevents particles of stripped down plastic bottles from entering the processing system. The frozen chunks of donor milk are thawed, using a slow, continuous heat with a mild churning action. Once thawed, a nutritional analysis is performed to determine specific nutritional levels of the pooled donor milk. Depending upon the desired human milk formulation, the system automatically adjusts the formulation, using validated sources from human milk origin, if augmentation above the levels of the donor milk is desired. Fortification at this point is limited to nutrients that are not adversely affected by heat. As the fortification is being done, the milk is gently churned. The pasteurization process takes place, again, in the same closed system, using the Holder Method of 62.5° C. for 20 minutes of 56° C. for 30 minutes. After pasteurization, the milk is cooled. Second stage fortification occurs at this point, with the addition of previously processed immunoglobulins, as well as selected, 100% screened human milk cells. After processing, final testing is done to determine that the pasteurization process has been successful.

The formulations and methods of the present invention may be embodied in other specific forms without departing from the teachings or essential characteristics of the invention. The described embodiments are therefore to be considered in all respects as illustrative and not restrictive. The scope of the present invention is defined in the following claims, rather than the previous description, and all changes that come within the meaning and range of equivalency of the claims are therefore to be embraced therein.

### Example 8

### Integration of the Collection and Distribution Over a Computer Network

The present invention specifies a method for facilitating the collection and distribution of human milk over a computer network. A provider will be qualified to participate based on an interview with the female provider. Upon initial qualification, a new file will be opened by the milk collection center that will include all qualification information obtained by the interview and the provider will be issued a unique provider code. The provider will then undergo a physical examination by a physician who will transmit the results of the physical examination to the milk collection company. The data from the physical examination will then be entered into the providers file. If the provider meets the qualifications of both the interview and physical examination, a reference sample of the providers milk is sent to a reference laboratory for testing. The reference laboratory will conduct biological testing on the sample including, DNA testing, viral and bacterial analysis, analysis of caloric content, protein content, IgG content and the content of a variety of drugs and pathogens. The data collected from the reference laboratory will be entered into the providers file in the milk collection company's database.

If the provider is qualified to participate following the tests by the reference laboratory, the unique provider code will then be affixed to empty milk containers and the empty milk containers will be shipped to the qualified provider. The provider, upon receipt of the empty containers will express milk into the containers and ship the filled containers back to the milk collection facility. Once at the milk collection facility, the milk is again tested for pathogenic and drug contaminants, and for nutritional content. The milk may then be concentrated or modified by addition of a human milk fortifier to achieve approximately about 3-10 kcal/oz and about 1-6 grams of protein/100 mL. The milk will then either be returned to the provider for use in the providers own infant or will be shipped to a different mother for use in feeding a different infant.

What is claimed:

1. A method for facilitating the collection and distribution of human milk over a computer network among a human milk provider, a milk company and a milk distribution center comprising the steps of:
(a) qualifying a human milk provider to participate;
(b) providing a provider account code to said provider wherein said provider account code corresponds to qualification information for the milk provider;
(c) receiving a filled first milk container bearing the provider account code from the milk provider;
(d) establishing a database for facilitating display of a plurality of descriptive line items of the filled milk containers, wherein each of said descriptive line items comprises identification of the milk, comprising the provider account code and/or said qualification information.
(e) filling a second milk container for shipment; and
(f) effecting the shipment of said second filled milk container to a distribution center.

2. The method of claim 1, further comprising the step of providing empty first milk containers to the milk provider.

3. The method of claim 1, wherein the provider account code is a bar code.

4. The method of claim 1, wherein the milk is tested for viral and bacterial contaminants.

5. The method of claim 1, implemented by a computer.

6. A computer program on a computer readable medium, comprising instructions to cause the computer to:
(a) provide a provider account code to a qualified human milk provider wherein the provider account code corresponds to qualification information for the qualified human milk provider;
(b) process a filled first milk container bearing the provider account code from the qualified human milk provider comprising associating a plurality of descriptive line items of the filled first milk container wherein the plurality of descriptive line items comprise said provider account code and/or said provider qualification information;
(c) fill a second milk container for shipment; and
(d) effect the shipment of the second filler milk container to a distribution center.

7. The computer program of claim 6, further comprising causing the identification of a qualified milk provider and effecting shipment of empty first milk containers to said qualified human milk provider.

8. The computer program of claim 6, wherein the provider account code is a bar code.

9. The computer program of claim 6, further comprising testing the milk for viral and bacterial contaminants.

10. A method for facilitating the collection and distribution of human milk utilizing a computer network comprising:
(a) qualifying a human milk provider and recording qualification information;
(b) providing a provider account code wherein said provider account code corresponds to the qualification information for the provider;
(c) receiving a filled first milk container bearing the provider account code from the provider;
(d) establishing a database for facilitating display of a plurality of descriptive line items of the first filled milk container, wherein each of said descriptive line items comprises the qualification information and/or provider account code;
(e) processing the human provider's milk, wherein the processing comprises analyzing the milk to determine the nutritional value of the milk;
(f) modifying the processed provider's milk from step (e) by adjusting the nutritional value of the milk; and
(g) effecting shipment of the processed provider's milk to a human recipient.

11. The method of claim 10, further comprising the step of providing empty first milk containers to the human provider.

12. The method of claim 10, wherein the human recipient is the human provider's own infant.

13. The method of claim 10, wherein the modifying further comprises altering the nutritional value of the milk by adding one or more nutrients to the milk.

14. The method of claim 10, wherein the provider account code is a bar code.

15. The method of claim 10, wherein the processing further comprises testing for viral and bacterial contaminants.

16. The methods of claim 10, wherein the provider code is associated with both the provider and the recipient.

17. The method of claim 10, implemented by a computer.

18. A computer program on a computer readable medium, comprising instructions to cause a computer to:
(a) provide a provider account code to a human milk provider wherein the provider account code corresponds to qualification information for the qualified human provider;
(b) process a filled first milk container bearing the provider account code from the qualified provider comprising associating a plurality of descriptive line items of the filled milk container including qualification information and/or provider account code, wherein the processing comprises analyzing the milk to determine the nutritional value of the milk;
(c) modify the milk by adjusting the nutritional value of the milk; and
(d) effect the shipment of the filled milk container containing the processed provider's milk to the provider, the provider's infant, a neonatal unit or a distribution center or.

19. The computer program of claim 18, wherein the provider account code is a bar code.

20. The method of claim 18, wherein the processing of the milk of step (b) further comprises testing the milk viral and/or bacterial contaminants.

21. The method of claim 18 wherein the adjusting of the nutritional value of the milk comprises adding human milk fortifier.

22. The method of claim 21, wherein adding said human milk fortifier increases the caloric and/or protein content of the provider's milk.

23. The method of claim 22, wherein the adding of the human milk fortifier increases the caloric content to about 3 to about 10 kcal/oz.

24. The method of claim 23, wherein the adding of the human milk fortifier increases the caloric content to about 4 kcal/oz.

25. The method of claim 23, wherein the adding of the human milk fortifier increases the caloric content to about 6 kcal/oz.

26. The method of claim 23, wherein the adding of the human milk fortifier increases the caloric content to about 8 kcal/oz.

27. The method of claim 23, wherein the adding of the human milk fortifier increases the caloric content to about 10 kcal/oz.

28. The method of claim 22, wherein the adding of the human milk fortifier increases the protein content to about 1 to about 6 grams of protein/100 mL.

29. The method of claim 28, wherein the adding of the human milk fortifier increases the protein content about 2 grams of protein/100 mL.

30. The method of claim 28, wherein the adding of the human milk fortifier increases the protein content to about 4 grams of protein/100 mL.

31. The method of claim 28, wherein the adding of the human milk fortifier increases the protein content to about 6 grams of protein/100 mL.

32. The method of claim 18 wherein the adjusting of the nutritional value of the milk comprises adding human milk fortifier.

33. The method of claim 32, wherein adding said human milk fortifier increases the caloric and/or protein content of the provider's milk.

34. The method of claim 34, wherein the adding of the human milk fortifier increases the caloric content to about 3 to about 10 kcal/oz.

35. The method of claim 33, wherein the adding of the human milk fortifier increases the caloric content to about 4 kcal/oz.

36. The method of claim 33, wherein the adding of the human milk fortifier increases the caloric content to about 6 kcal/oz.

37. The method of claim 33, wherein the adding of the human milk fortifier increases the caloric content to about 8 kcal/oz.

38. The method of claim 33, wherein the adding of the human milk fortifier increases the caloric content to about 10 kcal/oz.

39. The method of claim 28, wherein the adding of the human milk fortifier increases the protein content to about 1 to about 6 grams of protein/100 mL.

40. The method of claim 39, wherein the adding of the human milk fortifier increases the protein content to about 2 grams of protein/100 mL.

41. The method of claim 39 wherein the adding of the human milk fortifier increases the protein content to about 4 grams of protein/100 mL.

42. The method of claim 39, wherein the adding of the human milk fortifier increases the protein content to about 6 grams of protein/100 mL.
